(19) United States
(12) Patent Application Publication
(10) Pub. No.: US 2009/0258863 A1
(43) Pub. Date: Oct. 15, 2009

(54) Method for treatment of amyotrophic lateral sclerosis using talampanel

(76) Inventors: Miri Ben-Ami, Petah-Tikva (IL); Merav Bassan, Netanya (IL); Ella Sorani, Kadima (IL)

Correspondence Address: COOPER & DUNHAM, LLP, 30 Rockefeller Plaza, 20th Floor, NEW YORK, NY, 10112, US

(21) Appl. No.: 12/384,934

(22) Filed: Apr. 10, 2009

Related U.S. Application Data
(60) Provisional application No. 61/123,980, filed on Apr. 11, 2008.
(60) Provisional application No. 61/128,527, filed on May 21, 2008.

(51) Int. Cl. A61K 31/551

(52) U.S. Cl. 514/220

(57) ABSTRACT

A method of treating a human patient afflicted with amyotrophic lateral sclerosis (ALS) comprising periodically administering to the human patient for a therapeutically effective duration a pharmaceutical composition comprising an amount of talampanel therapeutically effective to provide a benefit to the human patient, thereby treating the human patient.

This application claims the benefit of U.S. Provisional Patent Application Nos. 61/123,980, filed Apr. 11, 2008 and 61/128,527, filed May 21, 2008, the contents of each of which are hereby incorporated by reference in their entirety.

Throughout this application various publications are referenced in full in parentheses. The disclosures of these publications in their entireties are hereby incorporated by reference into this application in order to more fully describe the state of the art to which this invention pertains.

### FIELD AND BACKGROUND OF THE INVENTION

Amyotrophic lateral sclerosis (ALS) is a motor neuron disease. It is characterized by degeneration and loss of upper (motor cortex) and lower (spinal cord and brainstem) motor neurons. Its clinical manifestations are related to the initial site of involvement: two-thirds of patients present with limb-onset ALS, characterized by asymmetric extremity weakness and wasting, while one third have bulbar-onset, with dysarthria, dysphagia and emotional lability (uncontrollable laughter, crying or smiling). Median survival time is about 3-5 years with death most often due to respiratory failure. Prognostically unfavorable factors are older age at time of onset of symptoms, short time from first symptoms to diagnosis, bulbar onset and worsening respiratory function (Brown H R., Amyotrophic lateral sclerosis and other motor neuron diseases. Braunwald E, Fauci A S, Kasper D L, Hauser S L, Longo D L, Jameson J L, Harrison's Principles of Internal Medicine. New York: McGraw-Hill professional, 2001, 2412-2416), (Strong M., Rosenfeld J., Amyotrophic lateral sclerosis: a review of current concepts. ALS and other motor neuron disorders, 2003, 4, 136-143), (Leigh P N., Baseline disease characteristics. ALS and other motor neuron disorders 2004, 5 (suppl 1), 64-67).

Incidence rate estimates of ALS range from 1-3 per 100,000 persons per year. In all populations ALS primarily affects the older population (>50 years of age; median age of onset approximately 60 years) (Leigh P N, Swash M, Iwasaki Y, et al., Amyotrophic lateral sclerosis: a consensus viewpoint on designing and implementing a clinical trial. ALS and other motor neuron disorders 2004, 5, 84-98). The disease has a male to female ratio of 1.6:1. Disease prevalence is estimated at 5-9 per 100,000 worldwide, though a rare form of ALS (associated with Parkinsonism and dementia) has been noted with higher prevalence in the western pacific, primarily Guam (McGeer P L, Schwab C, McGeer E, et al., Familial nature and continuing morbidity of the amyotrophic lateral sclerosis-parkinsonism dementia complex of Guam. Neurology 1997, 9, 400-409). Most cases of ALS are sporadic (SALS), 5% to 10% are inherited in an autosomal dominant manner as familial ALS (FALS) (Brown H R., Amyotrophic lateral sclerosis and other motor neuron diseases. Braunwald E, Fauci A S, Kasper D L, Hauser S L, Longo D L, Jameson J L, Harrison's Principles of Internal Medicine. New York: McGraw-Hill Professional, 2001, 2412-2416).

The pathophysiology leading to motor neuron degeneration in ALS is incompletely understood. Multiple mechanisms including mitochondrial dysfunction, excessive oxidative damage, alterations in the metabolism of glutamate and excessive activation of microglial cells are involved (Rothstein J D., Excitotoxic mechanisms in the pathogenesis of amyotrophic lateral sclerosis. Adv Neurol 1995, 68, 7-20). It is also evident that degenerating neurons are exposed to inflammation. Inflammatory cells, cytokines and components of the complement cascade are seen in the CNS of people with sporadic or familial ALS as well as in the SuperOxide Dismutase (SOD) 1 ALS mouse model. This immune response leads to the accumulation of pro-inflammatory mediators and free radicals that likely contribute to oxidative stress damage and subsequent neurodegeneration⁷ (Harrop J S, Sharan A D, Vaccaro A R, Przybylski G J., The cause of neurologic deterioration after acute cervical spinal cord injury. Spine 2001, 26, 340-346).

The excitatory neurotransmitter, glutamate, is widely distributed in the mammalian CNS. An excess of glutamate in the extracellular environment has been shown to result in neuronal death (Roy et al, 1998). The process is known as ‘excitotoxicity’, and is based on altered extracellular concentrations of glutamate. Normally, low levels of glutamate are maintained in the extracellular environment by active uptake mechanisms present on neurons and glia (Rothstein et al, 1996; Rothstein et al, 1992; Rothstein et al, 1993). However, in areas of the cortex affected in ALS there is evidence of reduced levels of expression of the glial glutamate transporter and reduced glutamate uptake activity (Rothstein et al, 1992).

Several lines of evidence suggest a role for glutamate in the pathogenesis of ALS (Rothstein et al, 1993). Increased glutamate levels have been demonstrated in the CSF of ALS patients. Reduced glutamate transport was found in synaptosomes derived from spinal cord and motor cortex of ALS patients, but not other brain areas (Rothstein et al, 1993). In an organotypic spinal cord slice model, reduction in glutamate uptake results in motor neuron death (Rothstein et al, 1993) and a variety of glutamate antagonists inhibit this process (Rothstein et al, 1993; Bilak et al, 2001; Drachman et al, 2000; Ho et al, 2000). Motor neurons appear to be particularly vulnerable to excitotoxic effects of glutamate and this is mediated by the glutamate AMPA receptor. The selective susceptibility of motor neurons may be due to the fact that the AMPA glutamate (GluR2) receptor subunits serve as gatekeepers for motor neuron survival preventing calcium influx. Evidence has linked sporadic ALS with the failure to edit key residues in ionotropic glutamate receptors (Kawahara et al, 2004) resulting in excessive influx of calcium ions into motor neurons which in turn triggers cell death (Carriedo et al, 2000; reviewed in Hugon et al, 1996).

The complex pathophysiology of ALS presents many potential therapeutic targets. However, although a wide range of agents has been investigated, only Rilutek®, an inhibitor of glutamate release, has demonstrated consistent benefit, and as of 2009 it is the only approved drug for the treatment of the disease (Miller R G, Mitchell J D, Lyon M, Moore D H, Rilutek® for amyotrophic lateral sclerosis (ALS) (motor neuron disease (MND). Cochrane database Syst Rev. 2002, 2, CD001447.), (Bensimon G. Lacomblez L, Meininger V., A controlled trial of Rilutek® in amyotrophic lateral sclerosis. N Eng J Med 1994, 330, 585-591), (Lacomblez L, Bensimon G, Leigh P N, Guillet P, Meininger V., Dose-ranging study of Rilutek® in amyotrophic lateral sclerosis. Lancet 1996, 347, 1425-1431). According to the Physician's Desk Reference, Rilutek® extends survival and/or time to tracheostomy; however, no mention is made of improvement of measures of function. Therefore, at present, ALS remains a disease for which limited effective treatment options are available. There is clearly an unmet need for additional more beneficial agents.

### SUMMARY OF THE INVENTION

This invention provides a method of treating a human patient afflicted with amyotrophic lateral sclerosis (ALS) comprising periodically administering to the human patient for a therapeutically effective duration a pharmaceutical composition comprising an amount of talampanel therapeutically effective to provide a benefit to the human patient.

This invention also provides a method of treating a symptom of amyotrophic lateral sclerosis (ALS) in a human patient afflicted with ALS comprising periodically administering to the human patient for a therapeutically effective duration a pharmaceutical composition comprising an amount of talampanel therapeutically effective to alleviate the symptom of ALS in the human patient.

This invention additionally provides a method for reducing the rate of functional decline in a human patient afflicted with amyotrophic lateral sclerosis (ALS) comprising periodically administering to the human patient for a therapeutically effective duration a pharmaceutical composition comprising an amount of talampanel (talampanel) therapeutically effective to reduce the rate of functional decline in the human patient.

This invention further provides a use of a pharmaceutical composition comprising a therapeutically effective amount of talampanel for the manufacture of a medicament for use in reducing the rate of functional decline in a human patient afflicted with amyotrophic lateral sclerosis (ALS) wherein such medicament is periodically administered to the human patient for a therapeutically effective duration.

This invention also provides a pharmaceutical composition for use in reducing the rate of functional decline in a human patient afflicted with amyotrophic lateral sclerosis (ALS) comprising a therapeutically effective amount of talampanel by periodic administration for a therapeutically effective duration.

### DETAILED DESCRIPTION OF THE INVENTION

This invention provides a method of treating a human patient afflicted with amyotrophic lateral sclerosis (ALS) comprising periodically administering to the human patient for a therapeutically effective duration a pharmaceutical composition comprising an amount of talampanel therapeutically effective to provide a benefit to the human patient, thereby treating the human patient.

In one embodiment, this invention provides a method wherein the benefit is a reduction in the rate of decrease in the ALSFRS-R score, the Manual Muscle Testing (MMT) score, the Slow Vital Capacity (VC) percent predicted value, the ALS-Specific Quality of Life (ALSSQoL) score, EuroQol-5 Dimensions (EQ-5D) Health Outcomes Scale score.

In a further embodiment, this invention provides a method wherein the benefit is a reduction in the rate of increase in the Zarit Burden Interview (ZBI) score.

In another embodiment this invention provides a method, wherein the benefit is a reduction in the rate of decrease in the Manual Muscle Testing (MMT) score, the Slow Vital Capacity (VC) percent predicted value, ALS-Specific Quality of Life (ALSSQoL) score, EuroQol-5 Dimensions (EQ-5D) Health Outcomes Scale score.

This invention also provides a method of treating a symptom of amyotrophic lateral sclerosis (ALS) in a human patient afflicted with ALS comprising periodically administering to the human patient for a therapeutically effective duration a pharmaceutical composition comprising an amount of talampanel therapeutically effective to alleviate the symptom of ALS in the human patient.

In one embodiment, this invention provides a method wherein the symptom of ALS is measured by the ALSFRS-R scale, the Manual Muscle Testing (MMT) grading system, the Slow Vital Capacity (VC) assay, the ALS-Specific Quality of Life (ALSSQoL) scale, EuroQol-5 Dimensions (EQ-5D) Health Outcomes Scale, Health Care Resource Utilization Questionnaire for ALS scale or Caregiver burden questionnaire, Zarit Burden Interview (ZBI) scale.

In another embodiment this invention provides a method wherein the symptom of ALS is measured by the Manual Muscle Testing (MMT) grading system, the Slow Vital Capacity (VC) assay, the ALS-Specific Quality of Life (ALSSQoL) scale, EuroQol-5 Dimensions (EQ-5D) Health Outcomes Scale, Health Care Resource Utilization Questionnaire for ALS scale or Caregiver burden questionnaire, Zarit Burden Interview (ZBI) scale.

In one embodiment, this invention provides a method wherein the rate of decrease in, or the symptom as measured by, the ALSFRS-R scale, is reduced by at least 15% as compared to a human patient not administered the pharmaceutical composition.

In another embodiment this invention provides a method wherein the benefit or the symptom is measured by an ALS validated outcome measure assessment protocol.

In another embodiment this invention provides a method wherein the duration of administration is greater than thirty-six weeks.

In a further embodiment this invention provides a method wherein the duration of administration is fifty-two weeks.

In yet another embodiment this invention additionally provides a method wherein the periodic administration is three times per day.

In an additional embodiment this invention also provides a method wherein the therapeutically effective amount of talampanel is 25 mg.

In an embodiment this invention yet further provides a method wherein the therapeutically effective amount of talampanel is 50 mg.

In another embodiment this invention provides a method wherein the therapeutically effective amount of talampanel is 75 mg/day.

In an additional embodiment this invention provides a method wherein the therapeutically effective amount of talampanel is 150 mg/day.

In another embodiment this invention also provides a method further comprising escalating a total daily dose administered to the human patient.

In a further embodiment, this invention additionally provides a method wherein the total daily dose is escalated from 37.5 mg to 75 mg.

In yet a further embodiment, this invention additionally provides a method wherein the escalating step comprises administering 37.5 mg total daily dose from baseline visit to week 1, 50 mg total daily dose from week 1 to week 2, 62.5 mg total daily dose from week 2 to week 3, and 75 mg total daily dose from week 3 and thereafter.

In another embodiment this invention provides a method wherein during the period from baseline visit to week 1 12.5 mg of talampanel is administered at each a.m., mid-day, and p.m.

In another embodiment this invention also provides a method wherein during the period from week 1 to week 2 12.5 mg of talampanel is administered at each a.m. and mid-day, and 25 mg at p.m.

In another embodiment this invention additionally provides a method wherein during the period from week 2 to week 3 25 mg of talampanel is administered at a.m., 12.5 mg at mid-day, and 25 mg at p.m.

In another embodiment this invention further provides a method wherein from week 3 and thereafter 25 mg of talampanel is administered at each a.m., mid-day, and p.m.

In an embodiment this invention also provides a method wherein the total daily dose is escalated from 75 mg to 150 mg.

In an embodiment this invention also provides a method wherein the escalating step comprises administering 75 mg total daily dose from baseline visit to week 1, 100 mg total daily dose from week 1 to week 2, 125 mg total daily dose from week 2 to week 3, and 150 mg total daily dose from week 3 and thereafter.

In another embodiment this invention provides a method wherein during the period from baseline visit to week 1 25 mg of talampanel is administered at each a.m., mid-day, and p.m.

In another embodiment this invention also provides a method wherein during the period from week 1 to week 2 25 mg of talampanel is administered at each a.m. and mid-day, and 50 mg at p.m.

In another embodiment this invention additionally provides a method wherein during the period from week 2 to week 3 50 mg of talampanel is administered at a.m., 25 mg at mid-day, and 50 mg at p.m.

In another embodiment this invention further provides a method wherein from week 3 and thereafter 50 mg of talampanel is administered at each a.m., mid-day, and p.m.

In another embodiment this invention provides a method wherein the escalating step is performed over a period of 4 to 6 weeks.

In yet another embodiment this invention also provides a method wherein the escalating step is performed over a period of 12 weeks.

In a further embodiment this invention also provides a method wherein the total individual dose (t.i.d.) is 25 mg.

In a further embodiment this invention also provides a method wherein the total individual dose (t.i.d.) is 50 mg.

In another embodiment this invention additionally provides a method wherein the talampanel is administered in 12.5 mg unit dose form.

In an embodiment this invention also provides a method wherein the talampanel is administered in 25 mg unit dose form.

In one embodiment this invention additionally provides a method wherein the rate of decrease is reduced as measured by an analysis of the change in Manual Muscle Testing (MMT) score over time as compared to a human patient not administered the pharmaceutical composition.

In another embodiment this invention also provides a method wherein the rate of decrease is measured by an analysis of the change in the Slow Vital Capacity (VC) percent predicted value over time as compared to a human patient not administered the pharmaceutical composition.

In an embodiment this invention additionally provides a method wherein the talampanel is administered as monotherapy.

In another embodiment this invention further provides a method further comprising the administration of another drug for the treatment of ALS.

In an additional embodiment this invention yet further provides a method further comprising the administration of another drug other than 2-amino-6-trifluoromethoxybenzothiazole for the treatment of ALS.

This invention provides a method for reducing the rate of functional decline in a human patient afflicted with amyotrophic lateral sclerosis (ALS) comprising periodically administering to the human patient for a therapeutically effective duration a pharmaceutical composition comprising an amount of talampanel therapeutically effective to reduce the rate of functional decline in the human patient.

This invention also provides the use of a therapeutically effective amount of talampanel for the manufacture of a medicament for use in periodically administering for a therapeutically effective duration to treat a human patient afflicted with amyotrophic lateral sclerosis (ALS).

This invention also provides the use of a therapeutically effective amount of talampanel for the manufacture of a medicament for use in periodically administering for a therapeutically effective duration to treat a symptom of amyotrophic lateral sclerosis (ALS) in a human patient afflicted with amyotrophic lateral sclerosis (ALS).

This invention also provides the use of a therapeutically effective amount of talampanel for the manufacture of a medicament for use in reducing the rate of functional decline in a human patient afflicted with amyotrophic lateral sclerosis (ALS) wherein such medicament is periodically administered to the human patient for a therapeutically effective duration.

This invention additionally provides a pharmaceutical composition for use in treating a human patient afflicted with amyotrophic lateral sclerosis (ALS) comprising a therapeutically effective amount of talampanel by periodic administration for a therapeutically effective duration to provide a benefit to the human patient.

This invention additionally provides a pharmaceutical composition for use in treating a symptom of amyotrophic lateral sclerosis (ALS) in a human patient afflicted with amyotrophic lateral sclerosis (ALS) comprising a therapeutically effective amount of talampanel by periodic administration for a therapeutically effective duration to alleviate the symptom of ALS in the human patient.

This invention additionally provides a pharmaceutical composition for use in reducing the rate of functional decline in a human patient afflicted with amyotrophic lateral sclerosis (ALS) comprising a therapeutically effective amount of talampanel by periodic administration for a therapeutically effective duration.

In one embodiment of the invention, the therapeutically effective amount of talampanel is 12 to 100 mg/administration; or 14 to 90 mg/administration; or 16 to 80 mg/administration; or 18 to 70 mg/administration; or 20 to 60 mg/administration; or 22 to 50 mg/administration; or 24 to 40 mg/administration; or 25 mg/administration; or 50 mg/administration. By a therapeutically effective amount of talampanel it is meant that all tenth and integer values within the range are specifically disclosed as part of the invention. Thus, 12.1, 12.2 . . . 99.8, 99.9; 13, 14 . . . 98, 99 mg/administration is included as embodiments of this invention.

In another embodiment, the escalating step is performed over a period of 4 to 12 weeks; or over a period of 4 weeks; or over a period of 5 weeks; or over a period of 6 weeks; or over a period of 7 weeks; or over a period of 8 weeks; or over a period of 9 weeks; or over a period of 10 weeks; or over a period of 11 weeks; or over a period of 12 weeks.

In another embodiment, the therapeutically effective amount of talampanel is in the range from 30 to 120 mg/day; or 40 to 110 mg/day; or 50 to 100 mg/day; or 60 to 90 mg/day; or 70 to 80 mg/day; or 75 mg/day. Alternatively, the therapeutically effective amount of talampanel is in the range from 70 to 200 mg/day; or 85 to 191 mg/day; or 100 to 182 mg/day; or 115 to 173 mg/day; or 130 to 164 mg/day; or 145 to 155 mg/day; or 150 mg/day. By a therapeutically effective amount of talampanel it is meant that all tenth and integer values within the range are specifically disclosed as part of the invention. Thus, 30.1, 30.2 . . . 199.8, 199.9; 31, 32 . . . 198, 199 mg/day is included as embodiments of this invention.

In yet another embodiment, the therapeutically effective amount of talampanel is in the range from 200 to 600 mg/week; or 260 to 590 mg/week; or 320 to 580 mg/week; or 380 to 570 mg/week; or 440 to 560 mg/week; or 500 to 550 mg/week; or 525 mg/week. Alternatively, the therapeutically effective amount of talampanel is in the range from 500 to 1110 mg/week; or 608 to 1100 mg/week; or 716 to 1090 mg/week; or 824 to 1080 mg/week; or 932 to 1070 mg/week; or 1040 to 1060 mg/week; or 1050 mg/week. By a therapeutically effective amount of talampanel it is meant that all tenth and integer values within the range are specifically disclosed as part of the invention. Thus, 200.1, 200.2 . . . 1109.8, 1109.9; 201, 202 . . . 1108, 1109 mg/week is included as embodiments of this invention.

In an embodiment of the invention, the rate of functional decline is reduced by at least 15%; or by at least 20%; or by at least 25%; or by at least 30%; or by at least 35%; or by at least 40%; or by at least 45%; or by at least 50%.

In yet another embodiment, the periodic administration of talampanel is effected daily. In another embodiment, the periodic administration of talampanel is effected twice daily at one half the amount. In another embodiment, the periodic administration of talampanel is effected three times daily at one third the amount. In an additional embodiment, the periodic administration of talampanel is effected once every 3 to 11 days; or once every 5 to 9 days; or once every 7 days; or once every 24 hours.

The administration of talampanel is oral, nasal, pulmonary, parenteral, intravenous, intra-articular, transdermal, intradermal, subcutaneous, topical, intramuscular, rectal, intrathecal, intraocular, buccal, by gavage, or via a feeding tube. The preferred route of administration for talampanel is oral. One of skill in the art would recognize that doses at the higher end of the range are required for oral administration.

In another embodiment, the dosage regimen of talampanel is administered to a patient based on a pharmacogenetic profile. In yet another embodiment, the dosage regimen of talampanel is administered to a patient based on a pharmacokinetic profile.

The specific embodiments and examples described herein are illustrative, and many variations can be introduced on these embodiments without departing from the spirit of the disclosure or from the scope of the appended claims. Elements and/or features of different illustrative embodiments and/or examples may be combined with each other and/or substituted for each other within the scope of this disclosure and appended claims.

### DEFINITIONS

### Baseline

In order to establish an initial data point for subsequent escalation of dosages, the half-maximal dose amount of the study is administered to patients during week 0 prior to incremental dose increases.

### Primary Endpoint:

Slope of the Changes from Baseline to Each Visit (In-Clinic and Telephonically) in ALSFRS-R Score

The primary end-point of this study is based on the rate of change (slope) from baseline to each visit (in-clinic and telephonically) in functional decline as measured by the ALSFRS-R score for monitoring the progression of disability in patients with ALS to each visit. All subjects who have at least one post-baseline ALSFRS-R measurement is included in the analysis.

A total of 559 subjects, randomized to one of three treatment groups, with a randomization ratio of 2:1:2 (Placebo, 25 mg t.i.d. talampanel and 50 mg t.i.d. talampanel, respectively) provides a power of 80% (at a two-sided 5% significance level) to detect a decrease of at least 20% in the slope of ALSFRS-R in the 50 mg t.i.d. talampanel compared to Placebo (assuming a deterioration rate in the Placebo arm of −1.03, SD=0.76 unit/month).

ALS Functional Rating Scale-Respiratory (ALSFRS-R)
The ALSFRS-R is a questionnaire based scale for monitoring the progression of disability in patients with ALS. The scale has been validated in ALS and the score changes linearly through at least the first year of the disease. The scale has also been shown to be a predictor of survival, and to correlate with muscle strength and pulmonary function (Cedarbaum J, Stambler N, Performance of the ALS Functional Rating Scale in multicenter clinical trials. J Neural Sci. 1997, 152 (Suppl), 1-9), (Cedarbaum J, Stambler N, Performance of the ALS Functional Rating Scale in multicenter clinical trials. J Neurol Sci. 1997, 152 (Suppl), 1-9). The components of the scale group into 4 domains that encompass gross motor tasks, fine motor tasks, bulbar functions and respiratory function. Altogether there are 12 items, each of which is scored between 0 and 4. Therefore, the total score ranges from 0 to 48. The higher the score, the less disabled the patient. The ALSFRS-R scale may be administered telephonically (Kasarskis E, Dempsey-Hall L, Thompson M M, Lu Le, et al., Rating the severity of ALS by caregivers over the phone using the ALSFRS-R. Amyotrophic Lateral Sclerosis Other Motor Neuron Disorders 2005, 6, 50-54.). The ALSFRS-R is evaluated by the investigator or by the coordinator at the in-clinic study visits: screening, baseline, and weeks 4/6, 12, 26, 40, and 52. In addition, the ALSFRS-R is administered telephonically at weeks 8, 17, 22, 31, 36, 44 and 48. If an unscheduled visit is conducted by telephone, the investigator may elect to administer the ALSFRS-R at that visit.

### Secondary Endpoint:

Time from Baseline to the First Occurrence of Either Death, Tracheostomy or Permanent Assisted Ventilation

The secondary efficacy endpoint is the time from baseline to the first of either death, tracheostomy or permanent assisted ventilation. All subjects are assigned a time value according to the following:
a) Subjects, who died, underwent tracheostomy or had permanent assisted ventilation, are assigned a value equal to the time from baseline to the first of these events.
b) Subjects who did not die and who required neither a tracheostomy nor permanent assisted ventilation during the study are assigned a right-censored value equal to the time from baseline to their study termination.

Time from baseline to the first occurrence of either death, tracheostomy or permanent assisted ventilation compares the 50 mg t.i.d. talampanel group and placebo group using the Cox's proportional hazard model (SAS® PHREG Procedure). The model includes the following terms: treatment group (placebo, 25 mg t.i.d. talampanel and 50 mg t.i.d. talampanel), center, riluzole (Rilutek®) use at baseline, site of ALS onset, time from ALS onset to baseline, baseline ALSFRS-R score, baseline slow VC and baseline body mass index (BMI). The contrast that compares the 25 mg talampanel group and placebo group is evaluated according to the hierarchy approach defined above. The Kaplan-Meier estimates for the first event out of either death, tracheostomy or permanent assisted ventilation are computed and graphically presented by treatment group.

Tracheostomy
Tracheostomy is defined as a surgical procedure on the neck to open a direct airway through an incision in the trachea.

Permanent Assisted Ventilation
Permanent Assisted Ventilation is defined as the use of any ventilation device more than 23 hours a day.

Slow Vital Capacity (VC)
Slow VC is a measure of lung function assessed by a spirometer. It is assessed during an expiratory maneuver to measure how an individual exhales volumes of air as a function of time. Starting from end-tidal volume the subject makes a full inspiration and subsequently exhales maximally. This represents the expiratory vital capacity (ECV), or slow vital capacity, the maximal volume of air exhaled from the point of maximal inhalation. The test is performed by experienced personnel (the investigator or coordinator). The result is compared to a reference value to generate a percent predicted value. The test is performed at least 3 times and no more than 5 times, and the best trial is recorded in the CRF. The Slow VC is assessed at all the scheduled in-clinic study visits. The investigator may elect to assess Slow VC if an unscheduled clinic visit occurs.

### Additional Endpoints:

In the analyses of the additional endpoints, each one of the active treatment arms (50 mg t.i.d. talampanel and 25 mg t.i.d. talampanel) is compared to the placebo group.

Change from Baseline to Last Observed Value (LOV) in ALSFRS-R

The change in ALSFRS-R from baseline to LOV is compared between the treatment groups using an analysis of covariance (ANCOVA, SAS® GLM procedure). Treatment group (placebo, 25 mg t.i.d. talampanel and 50 mg t.i.d. talampanel), center, riluzole use at baseline, age at baseline, site of ALS onset, time from ALS onset to baseline and baseline ALSFRS-R score is included in the model. The adjusted means of the 50 mg t.i.d. group and the 25 mg t.i.d. group are compared to placebo by performing two relevant single degree of freedom comparisons.

### Time to Death

All subjects are assigned a value equivalent to the period of time from baseline to one of the following events:
a) Subjects who died are assigned a value equal to the time from baseline to death.
b) Subjects who did not die are assigned a right-censored value equal to the time from baseline to termination.

The analysis compares the treatment groups using the Cox's proportional hazard model (SAS® PHREG Procedure). The model includes the following terms: treatment group (placebo, 25 mg t.i.d. talampanel and 50 mg t.i.d. talampanel, with placebo as the reference category), center, riluzole use at baseline, site of ALS onset, time from ALS onset to baseline, baseline ALSFRS-R score, baseline slow VC and baseline BMI. The Kaplan-Meier estimates for the death are computed and graphically presented by treatment group.

Slope of the Changes from Baseline to Each Visit in Pulmonary Function as Assessed by Slow VC

The statistical analysis is a Repeated Measures Analysis of covariance model using SAS® MIXED Procedure. The dependent variable is the Change from baseline in Slow VC at each post-randomization visit and the model which aims to compare the slopes of change in Slow VC between talampanel groups and placebo includes the following fixed effects: time from baseline, treatment group (placebo, 25 mg t.i.d. talampanel and 50 mg t.i.d. talampanel), time by treatment interaction, center, riluzole use at baseline, age at baseline, site of ALS onset, time from ALS onset to baseline and baseline Slow VC score. The individual subject intercept and time effect are included in the model as random effects (random intercept and slope model).

The statistical analysis is a comparison between the treatments groups' slopes, derived from the time by treatment interaction term in the model described above.

The linearity of the SVC over time is tested with the same methodology as described for the primary endpoint.

Change from Baseline to LOV in Slow VC

Change from baseline to LOV in Slow VC is compared between the treatment groups using an analysis of covariance (ANCOVA, SAS® GLM procedure). Treatment group (placebo, 25 mg t.i.d. talampanel and 50 mg t.i.d. talampanel), center, riluzole use at baseline, age at baseline, site of ALS onset, time from ALS onset to baseline and baseline Slow VC value is included in the model. The adjusted means of the 50 mg t.i.d. group and the 25 mg t.i.d. group are compared to placebo by performing two relevant single degree of freedom comparisons.

Slope of the Changes from Baseline to Each Visit in Manual Muscle Testing (MMT)

The statistical analysis is a Repeated Measures Analysis of covariance model using SAS® MIXED Procedure. The dependent variable is the Change from baseline in MMT at each post-randomization visit and the model which aims to compare the slopes of change in MMT between talampanel groups and placebo includes the following fixed effects: time from baseline, treatment group (placebo, 25 mg t.i.d. talampanel and 50 mg t.i.d. talampanel), time by treatment group interaction, center, riluzole use at baseline, age at baseline, site of ALS onset, time from ALS onset to baseline, and baseline MMT score. The individual subject intercept and time effect are also included in the model as random effects (random intercept and slope model).

The statistical analysis is a comparison between the treatments groups' slopes, derived from the time by treatment interaction term from the model described above.

The linearity of the MMT over time is tested with the same methodology as described for the primary efficacy.

Manual Muscle Strength Testing (MMT)
The MMT is a standard grading system derived from the Medical Research Council Scale in which the strength of a muscle is graded (Medical Research Council. Aide to the examination of the peripheral nervous system: London. Her Majesty's Stationary Office, 1976, 14). Each muscle is scored from 0 to 5, with 0 representing paralysis and 5, normal strength. The final MMT score is the sum of scores of the muscles tested. In this trial, 22 muscles are assessed 5 of the upper limb (left and right), 5 of the lower limb (left and right) and 2 neck muscles. Based on 22 muscles the final MMT score ranges from 0 (total incapacity) to 110 (normal). The MMT is performed by trained experienced personnel (the investigator, coordinator or physical therapist) at baseline, and at weeks 12, 26, 40 and 52. Effort is made to ensure that the same evaluator assess a subject for the entire study duration.
   Change from Baseline to LOV in MMT

Change from baseline to LOV in MMT is compared between the treatment groups using an analysis of covariance (ANCOVA, SAS® GLM procedure). Treatment group (placebo, 25 mg talampanel and 50 mg talampanel), center, riluzole use at baseline, age at baseline, site of ALS onset, time from ALS onset to baseline and baseline MMT is included in the model. The adjusted means of the 50 mg t.i.d. group and the 25 mg t.i.d. group are compared to placebo by performing two relevant single degree of freedom comparisons.

Change from Baseline to LOV in ALS-Specific Quality of Life (ALSSQoL) to Assess Drug Effect on Health Status [US and Canada Only]

Change from baseline to LOV in ALS-Specific Quality of Life (ALSSQoL) is compared between the treatment groups using an analysis of covariance (ANCOVA, SAS® GLM procedure). Treatment group (placebo, 25 mg t.i.d. talampanel and 50 mg t.i.d. talampanel), center, riluzole use at baseline, age at baseline, site of ALS onset, time from ALS onset to baseline and baseline ALSSQoL score is included in the model. The adjusted means of the 50 mg t.i.d. group and the 25 mg t.i.d. group are compared to placebo by performing two relevant single degree of freedom comparisons.

ALS-Specific Quality of Life (ALSSQoL) (US and Canada Only)
The ALSSQoL is a 46-item, validated disease-specific questionnaire. It covers the following six health concepts:
a. Negative emotion
b. Intimacy
c. Physical symptoms
d. Interaction with people and the environment
e. Religiosity
f. Bulbar function
The ALSSQoL is being included because, unlike the SF-36®, it is less focused on strength and physical function and provides greater insight into factors that patients with ALS have said are important to their QoL.
Each item in the questionnaire is scored on a 1 to 10 scale with 1 the least desirable situation and 10 the most desirable (several items need to be transformed prior to calculating a score). An ALSSQoL score for each individual is defined as the sum of the scores divided by the number of questions answered by that individual. The ALSSQoL is assessed at baseline, and at the week 26 and 52/ET visits.
   Change from Baseline to LOV in Quality of Life EuroQol-5 Dimensions (EQ-5D) Health Outcomes Scale to Assess Drug Effect on Health Status

Each of the two scores derived from this questionnaire are analyzed in the following manner: Change from baseline to LOV in a score is compared between the treatment groups using an analysis of covariance (ANCOVA, SAS® GLM procedure). Treatment group (placebo, 25 mg t.i.d. talampanel and 50 mg t.i.d. talampanel), center, riluzole use at baseline, age at baseline, site of ALS onset, time from ALS onset to baseline and baseline score are included in the model. The adjusted means of the 50 mg t.i.d. group and the 25 mg t.i.d. group are compared to placebo by performing two relevant single degree of freedom comparisons.

Euro QoL-5 (EQ-5D) Health Outcomes Scale
The EQ-SD is composed of 2 parts: the first part is as-item standardized instrument that provides a simple descriptive profile and a single index value for health status across five dimensions:
a. Mobility
b. Self-Care
c. Usual Activities
d. Pain-Discomfort
e. Anxiety/Depression
The EQ-50 is important for the clinical and economic evaluation of a new treatment for a particular disease. It has been specifically designed to complement other outcome measures such as the SF-36® or disease-specific measures, like the ALSSQoL. The EQ-5D score can be computed as the sum of the 5 items.
The second part consists of a continuous visual analog scale which is scored separately and has the subject rating his/her own health on one dimension.
The EQ-5D is assessed at baseline and at the week 4-6, 12, 26, 40 and 52/ET visits. This questionnaire is completed by all 5 subjects.
   Change from Baseline to LOV in Health Care Resource Utilization Questionnaire for ALS to Assess Drug Effect on Health Care Resource Use

Changes from baseline in each of the items in this questionnaire are analyzed using descriptive statistics presented by treatment group.

Health Care Resource Utilization Questionnaire for ALS
The Health Care Resource Utilization Questionnaire for ALS is a 7-item instrument designed to ask about visits with health care professionals not related to this study as well as the use of non-health care services (i.e., paid care-giving) and dietary, walking and breathing aids that contribute to the economic cost of ALS. This questionnaire is important for the economic evaluation of a new treatment for ALS. Each of the questions in this questionnaire is analyzed separately.
The Health Care Resource Utilization Questionnaire for ALS is assessed at baseline and at the weeks 4-6, 12, 26, 40 and 52/ET visits. This questionnaire is completed by all subjects.
   Change from Baseline to LOV in Zarit Burden Interview (ZBI) to Assess Drug Effect on Caregiver Burden

Change from baseline to LOV in Zarit Burden Interview (ZBI) to assess drug effect on Caregiver Burden is compared between the treatment groups using an analysis of covariance (ANCOVA, SAS® GLM procedure). Treatment group (placebo, 25 mg t.i.d. talampanel and 50 mg t.i.d. talampanel), center, riluzole use at baseline, age at baseline, site of ALS onset, time from ALS onset to baseline and baseline ZBI score are included in the model. The adjusted means of the 50 mg t.i.d. group and the 25 mg t.i.d. group are compared to placebo by performing two relevant single degree of freedom comparisons.

Caregiver Burden Questionnaire, Zarit Burden:Interview (Zb:I) (to be Completed by Caregiver Only)
The ZBI is a 22-item validated scale that provides an assessment of caregivers' feelings and their responses to the demands of caring for someone with a disease like ALS. Having been used with other ALS caregivers, the ZBI is being included because ALS is a disease characterized by a rapidly progressive loss of function that requires assistance from caregivers. If a new treatment has an impact on care needs, it is important for the clinical and economic evaluation of that treatment. Each item response ranges from 0 to 4, where higher score indicates greater caregiver distress. A ZBI score for each individual is computed by summing items scores and dividing by number of questions answered by that individual.
The ZBI is assessed at baseline and at the weeks 12, 26, 40 and 52/ET visits. This questionnaire is completed by all caregivers who signed caregiver informed consent form at baseline visit.

As used herein, “reducing the rate of functional decline in a human patient” means slowing the progression of disability in a patient afflicted with ALS, as determined by anyone of the ALSFRS-R scale, by MMT grading, by the ALSSQoL score, by the EQ-5D score, by the Health Care Resource Utilization Questionnaire score, by the ZBI score, by VC measure, by time to death, by time to tracheostomy, by time to permanent assisted ventilation, or by any combination of each of the foregoing.

### Pharmacokinetics

It is recognized that pharmacokinetic variability within the population is an important contributory factor to inter-individual differences in drug pharmacodynamic response. Talampanel pharmacokinetic profile has been thoroughly characterized in several clinical studies in both healthy volunteers and epilepsy subjects. However, it is generally recommended to characterize the pharmacokinetic profile of compounds in development in the subjects with the disease of interest.

### Rich/Full Pharmacokinetics (PK)

Rich PK sampling allows a better definition of the basic parameters in the patient population which are important factors for performing the population PK analyses. Plasma samples are taken from a total of 25-30 subjects, preferably half of each sex, from pre-dose to 8 h post dose on a single occasion on week 4-6 visit. The samples are taken at pre-dose, 0.5, 1, 1.5, 2, 3, 4, 6, 8 hours post dose.

### Pharmacogenetics

It is recognized that genetic variation within the population can be an important contributory factor to inter-individual differences in drug distribution and response and can also serve as a marker for disease. Pharmacogenetics investigates the relationship between drug response and genetic differences. Pharmacogenetics ultimately allows the development of personalized medications based on the genotype of each subject to improve the drug efficacy and safety among subjects.

An association between a genetic polymorphism and clinical outcome helps to explain inter-individual variability in that outcome and helps to identify population subgroups that respond differently to a drug.

The objective of this pharmacogenetic study is to collect and store DNA samples for analyzing the polymorphism of N-Acetyltransferase NAT2 gene and to use in future exploratory analyses.

N-acetylation was identified as one metabolic pathway of talampanel, catalyzed by N-acetyltransferse NAT2 isozymes. A genetic polymorphism in the NAT2 gene affects the acetylation rate. N-acetylation of talampanel is highly dependent on NAT2 activity. In previous clinical trial, measurable levels of NAC talampanel were seen primarily in fast acetylator subjects, but not in slow acetylators. Fast acetylators possess two copies of the wild type allele NAT2*4 and also of NAT2*12 and NAT2*13, intermediate acetylators possess two mutated alleles. The three common slow acetylator alleles identified are NAT2*5A, NAT2*6A and NAT2*7A. The NAT2*5 alleles have the greatest reductions in metabolic activation while NAT2*6 alleles have more moderate reductions and NAT2*7 alleles have the smallest, but yet significant, reductions. The frequency of NAT2 slow acetylators (about 2-10% in Asian, 60-70% in Caucasians, 30-40% in Africans) and NAT2 fast acetylators (about 40-60% in Asian, 3-15% in Caucasian, 10-25% in Africans) varies markedly with ethnicity.

This research includes analyses that seek to define causal relationships between variation NAT2 gene and response to talampanel, as well, as serious adverse effects. Other genetic markers related to talampanel pharmacokinetics and pharmacodynamics might be also implemented in the study. Furthermore the exploration of unknown genetic polymorphisms related to the response and safety parameters might also be performed using a genome-wide association system.

To participate in the optional pharmacogenetic component of this study, subjects (or their legally acceptable representative) must have signed the informed consent form for pharmacogenetic research. Refusal to consent for this component does not exclude a subject from participation in the clinical study.

A blood sample (6 mL) is collected once at baseline from subjects who gave separate informed consent for the pharmacogenetic component of the study. If the sample is not collected on baseline, it may be collected at any other time during the conduct of the study. The samples are stored for a period of up to 15 years.

### EXAMPLES

### Example 1

### Evaluating the Efficacy, Tolerability and Safety of Oral Administration of Talampanel Compared to Placebo in Subjects with ALS

A multinational, multicenter, randomized, double-blind, placebo-controlled, parallel group study in subjects diagnosed with ALS.

### Methods

A subject is assessed for study eligibility at a screening visit (one to 4 weeks prior to baseline). Subjects who are found eligible to participate in the study are randomized in a 2:1:2 ratio into one of the following three treatment groups based on a randomization scheme with blocks stratified by center:
1. 50 mg (25 mg capsule×2) talampanel t.i.d. (150 mg daily)
2. 25 mg (12.5 mg capsule×2) talampanel t.i.d. (75 mg daily)
3. Placebo

After undergoing randomization subjects receive their first dose of study treatment on site. There are two possible escalation periods to reach maximal dose of 6 capsules per day, as follows:
Screening Period Up to 4 weeks but not less than 1 week. The period between screening visit and baseline visit. During this time period all relevant data for the assessment of subjects eligibility should be collected and reviewed i.e. lab results, ECG, medical history etc.
First Escalation Period: A period of 4 to 6 weeks, following screening period. This period starts at baseline visit, following randomization;
Second Escalation Period: A period of 2 weeks, this period starts at week 10 visit and ends at week 12 visit.
Maintenance Period: A time period of 46 to 48 weeks, following the first escalation period, starting after reaching maximal achieved dose in the first escalation period. During this period oral administration of talampanel 25 mg t.i.d., 50 mg t.i.d. (or maximal achieved dose) or matching oral placebo is maintained.

The first escalation period is a period of 4-6 weeks, following randomization. The dose escalation plan for the 25 mg and 50 mg t.i.d. dose levels, respectively, is as follows:
First week: from baseline visit to week 1 (telephone visit): Three capsules (37.5 mg or 75 mg daily) one each a.m., mid-day and p.m. dosing.
Second week: from week 1 (telephone visit) to week 2 (telephone visit): Four capsules (50 mg or 100 mg daily) one each a.m. & mid-day, two at p.m. dosing.
Third week: from week 2 (telephone visit) to week 3 (telephone visit): Five capsules (62.5 mg or 125 mg daily) two at a.m. one in mid-day and two at p.m. dosing.
Fourth week: from week 3 (telephone visit) to week 4 on site visit: Six capsules (75 mg or 150 mg daily) two at each a.m., mid-day and p.m. dosing.

During this dose escalation phase, subjects who are unable to tolerate the scheduled increase in dose due to adverse effects (AE), may have their dose reduced to the previous level. A weekly (weeks 1-3 or 1-5 if a dose escalation period of 6 weeks is needed) telephone call one to three days prior to dose escalation to the subject, to assess tolerability to drug is required. First dose escalation period should be 4 weeks but may continue for up to 6 weeks from baseline visit. The investigator can change the escalation scheme per his/her discretion while keeping the following limitations:
1. Not exceeding 6 capsules per day.
2. Not exceeding 2 capsules per administration.
3. Maintain intervals of at least 3 days between dose increases.

An additional telephone call should be performed in week 6, (only if in-clinic visit of end of first escalation period was performed in week 4) to assess tolerability to drug.

Subjects should be on maximal achieved dose at least 3 days prior to next in-clinic visit at the end of the escalation period.

If a subject could not reach the maximal dose (6 capsules per day) he/she is able to resume increase dose (second escalation period) starting at week 10 visit, to reach maximal dose of 6 capsules. After 11^{th} and 12^{th} week escalation period there are no additional attempts to increase dose. The maximal achieved dose for a subject should not exceed at any time after week 12 visit.

A mandatory phone call is performed at week 10 scheduled visit, prior to additional attempt to reach maximal dose. A mandatory phone call is also performed at the end of each week during escalation periods on study drug, one to three days prior to continuation of dose escalation.

All efforts should be made to reach minimal dose of 5 capsules per day. Patients, minimal dose at the end of the escalation periods should be no less than 5 capsules per day. In case the subject cannot tolerate 5 capsules or more the subject does not have to withdraw from the trial.

Scheduled study visits are conducted in-clinic at study weeks: screening (−4 to −1), baseline, end of first escalation period (4-6), 12, 26, 40, and 52/ET.

Telephone call visits to assess the revised ALS Functional Rating Scale (ALSFRS-R) and record any changes in adverse events and concomitant medications are conducted at study weeks: 8, 17, 22, 31, 36, 44, and 48.

At in-clinic visits the following are assessed:
Revised ALS Functional Rating Scale (ALSFRS-R) (screening, baseline, weeks 4-6, 12, 26, 40, 52/ET and unscheduled)
Slow VC (screening, baseline, weeks 4-6, 12, 26, 40, 52/ET and unscheduled)
Manual Muscle Testing (MMT) (baseline, weeks 12, 26, 40, 52/ET and unscheduled)
ALS-Specific Quality of life (ALSSQoL) scale (baseline, weeks 26, 52/ET) [US and Canada Only]
EuroQol-5 Dimensions (EQ-5D) health outcomes scale (baseline, weeks 4-6, 12, 26, 40 and 52/ET)
Health Care Resource Utilization Questionnaire for ALS (baseline, weeks 4-6, 12, 26, 40, 52/ET)
Caregiver burden questionnaire, Zarit Burden Interview (ZBI) [caregiver] (Baseline, weeks 12, 26, 40, 52/ET)
Laboratory (screening, baseline, weeks 4-6, 12, 26, 40, 52/ET and unscheduled)
ECG (screening, baseline (3 times), weeks 4-6, 12, 26, 40, 52/ET and unscheduled)
Physical examination (screening, baseline, weeks 4-6, 12, 26, 40 and 52/ET and unscheduled)
Vital signs and weights (screening, baseline, weeks 4-6, 12, 26, 40 and 52/ET and unscheduled)
Check of concomitant medications, AEs and compliance
Pharmacokinetics (PK): Safety Pharmacokinetics (PK) blood samples are taken after at least 3 days on the maximal achieved dose at end of first escalation period at week 4-6 (pre-dose, anytime in the range of 0.5-2 hours post-dose and anytime in the range of 2-4 hours post dose with at least 1 hour difference between the 2 post dose samples) and at week 12 (pre-dose, anytime in the range of 0.5-2 hours post-dose and anytime in the range of 2-4 hours post dose with at least 1 hour difference between the 2 post dose samples). Additional PK blood samples are taken at week 40 (pre-dose, anytime in the range of 0.5-2 hours post-dose and anytime in the range of 2-4 hours post dose with at least 1 hour difference between the 2 post dose samples).
   Any subjects exceeding the maximum talampanel plasma level (1200 ng/mL), as detected in one of the PK tests are excluded from further participation in the study.
   Full PK sampling is performed (week 4-6 at pre-dose, 0.5, 1, 1.5, 2, 3, 4, 6, 8 hours post dose, only in selected countries and sites).
Pharmacogenetic analysis: Blood sample for Pharmacogenetic analysis are taken at baseline visit.
The safety and tolerability of long term administration of talampanel in ALS patients is assessed using adverse events, laboratory data, vitals, ECG and the ability to remain on the assigned treatment.
Un-scheduled visits may be conducted at any time for safety reasons or for any other reason.

### Subject Population

This study includes subjects at an early clinical stage of disease to maximize the possibility to discern a clinical effect measured by functional scale.

For ethical considerations, subjects receiving riluzole (Rilutek®) are permitted to enter the study on condition that the dose is maintained stable for at least 8 weeks prior to screening. Riluzole is permitted during the study.

It is estimated that 15-20% change in the rate of decline in the ALSFRS-R produces a change that is clinically meaningful to patients. A total of 540 subjects, randomized to one of three treatment groups, with a randomization ratio of 2:1:2 (placebo, 25 mg t.i.d. talampanel and 50 mg t.i.d. talampanel, respectively) provide a power of 80% (at a two-sided 5% significance level) to detect a decrease of 20% in the slope of ALSFRS-R in the 50 mg t.i.d. talampanel compared to placebo (assuming a deterioration rate in the placebo arm of −1.03, SD=0.76 unit/month).

### Inclusion Criteria

Subjects must meet all inclusion criteria in order to be eligible for the study:
1. Males and females with definite, probable or probable laboratory-supported ALS according to the World Federation of Neurology revised El Escorial criteria. ALS may be familial or sporadic.
2. Subjects must be between 18 and 80 years of age (inclusive).
3. Subjects must have experienced their first ALS symptoms within 3 years inclusive prior to the screening visit.
4. Subjects must have a Slow VC score equal to or greater than 70% of the predicted value for gender, height and age at the screening and baseline visits.
5. The sum of the 3 respiratory items from the ALSFRS-R must total at least 10 points at the screening and baseline visits.
6. Subjects taking riluzole must be on a stable dose for at least 8 weeks prior to the screening visit.
7. Participants must be able to take oral medication at time of screening and baseline visits.
8. Subjects must be willing and able to give written informed consent prior to performing any study procedures. If the subject is unable to write, he/she may give oral consent or if not possible visual consent (such as head nodding) in the presence of at least one witness as provided in local country legislation.

### Exclusion Criteria

Any of the following conditions exclude the subject from entering the study:
1. The use of mechanical ventilation (invasive or non-invasive, including Continuous Positive Airway Pressure (CPAP) for any part of the day or night or Bilevel Positive Airway Pressure (BiPap) for any part of the day prior to the screening visit or baseline.
2. Feeding tube present at time of screening or baseline.
3. Any clinically significant or unstable medical or surgical condition including cardiovascular, hepatic, pulmonary, renal, autoimmune, endocrine, metabolic, malignancy or psychiatric or any other condition that, in the investigator's opinion, places the subject at undue risk by participating in the study.
4. Patients whose mean QTc value calculated from 3 baseline measurements is above 450 msec.
5. Patients with ECG signs of Brugada syndrome and/or complete or incomplete RBBB during screening and/or baseline visits
6. Patients with clinical signs and symptoms of dementia.
7. Known HIV positive.
8. History of known sensitivity or intolerability to benzodiazepines.
9. Subjects having used within the specified time prior to screening any of the following:
   Talampanel (any previous use)
   Mecasermin (rhIGF-1) (within 4 weeks prior to screening)
   Chronic use of minocycline (14 consecutive days or more within 4 weeks prior to screening)
   Chronic use of lithium carbonate use within 4 weeks prior to screening
   Use of more than 600 mg/day coenzyme Q10 (within 4 weeks prior to screening)
   Any marketed drug (within 12 weeks prior to screening) if its use was not clearly indicated for any underlying medical condition other than ALS (symptomatic drugs for ALS and supplements allowed)
   Subjects participating in any other investigational drug trial and use of any other investigational drug within 12 weeks prior to screening
   Taking any drugs which induce or inhibit talampanel metabolism within 2 weeks prior to screening
   Taking substrates of CYP2C8 within 2 weeks prior to screening, with the exception of amiodarone and chloroquine that should not be taken within 4 months prior to screening
10. Females who are pregnant or nursing.
11. Females of child-bearing potential who do not practice medically acceptable methods of contraception [surgical sterilization, IUD, hormonal preparations, or double barrier method (e.g. condom or diaphragm, and spermicide)].
12. Addiction to a drug or substance within the past year prior to screening.
13. Any condition which the investigator feels may interfere with participation in the study.
14. Subjects unable at time of the screening and baseline visits to comply with the planned schedule of study visits and study procedures.

### Talampanel Dose

A dosage regimen of talampanel at 25 mg t.i.d. (75 mg daily) and 50 mg t.i.d. (150 mg daily) is selected for this study based on safety and tolerability data accumulated from prior clinical trials and preliminary activity in a pilot study of talampanel in ALS. Previous clinical trials with talampanel have shown that adverse events are reduced when the dose is gradually increased over time. In this study, the daily dose is increased by 12.5 mg or 25 mg daily each week to reach 75 mg or 150 mg daily (25 mg t.i.d. or 50 mg t.i.d.) respectively, at the 4^{th} to 6^{th} week or at the 11^{th} to 12^{th} week if needed. The 150 mg daily (50 mg t.i.d.) dose has been found to be a safe and tolerated. Approximately 85% of the population is expected to tolerate this dose. The 75 mg daily (25 mg t.i.d.) dose is evaluated to determine minimal effective dose in the ALS population.

A pilot study of talampanel in ALS compared 50 mg t.i.d of talampanel to placebo. The study found a positive trend in the ALSFRS scores of talampanel treated relative to placebo (average score −7.1 vs. −10.2, p=0.08). Thus, the safety and the tolerability of 50 mg t.i.d considered along with the preliminary efficacy results at that dose all resulted in the selection of 50 mg t.i.d talampanel for the trial.

There is a concern that exposing subjects to talampanel plasma concentrations above 1200 ng/mL may result in adverse events. Although the dose selected for the ALS study (25 mg t.i.d and 50 mg t.i.d.) is not likely to result in plasma concentrations above the maximum specified, the measuring of talampanel plasma concentration at the time of anticipated steady state provides an additional assessment of safety for the subject population. In case subject's plasma level exceeds 1200 ng/mL the subject is excluded from further participation in the study.

### Results

### Primary Endpoint

Slope of the Changes from Baseline to Each Visit (In-Clinic and Telephonically) in ALSFRS-R Score (a Comparison Between 50 mg t.i.d. Talampanel and Placebo)

The primary endpoint is the slope of the changes from baseline to each visit (in-clinic and telephonically) in ALSFRS-R score.

A noticeable benefit is achieved as a reduction in the rate of functional decline from baseline to each visit as measured by the ALSFRS-R in the 50 mg t.i.d. talampanel treatment group.

Observations of all subjects who have at least one post-baseline ALSFRS-R measurement are analyzed. The analysis of the slopes of the ALSFRS-R, based on the Repeated Measures Analysis (RMA) of covariance model, comparing the ALSFRS-R slopes of change from baseline between 50 mg t.i.d. talampanel and Placebo, includes the following fixed effects: time from randomization, treatment group, time by treatment interaction, center, Rilutek® use, age, site of ALS onset, time from ALS onset and baseline ALSFRS-R score. The individual subject intercept and the time effects are also included in the model as random effects (random intercept and slope model). The principal statistical analysis is a comparison between the slopes of 50 mg t.i.d. talampanel and placebo, derived from the relevant contrast defined for the time by treatment interaction term from the RMA model described above. A statistically significant decrease of at least 15% in the slopes of ALSFRS-R is observed in the group treated with 50 mg t.i.d. talampanel as compared to the placebo group.

The principal analysis is based on the assumption of linear decline in ALSFRS-R within treatment group, a pattern that has been shown previously in other ALS studies. Nevertheless, both statistical testing and visual inspection of ALSFRS-R means is performed. To test if the relation between ALSFRS-R and time-in-trial is linear within treatment groups, a repeated measures ANCQVA model (SAS® MIXED procedure) is fitted, similar to the one proposed for the principal analysis, first with time as a continuous variable (representing a linear relation), and second, with time as a by-month categorical variable (representing a general relation). The log likelihood ratio test for nested models is used to test the contribution of the “general relation” beyond the “linear relation”. Statistically significant deviation from linearity is declared if the p-value of the chi-square statistic with the appropriate degrees of freedom is less than 0.05.

Due to the high sensitivity of this test, small deviations may yield statistical significance, although clinically irrelevant figures. Therefore, to determine if the deviation is clinically meaningful, a visual inspection of the mean changes in ALSFRS-R by time, separately for each group, is performed, as well as inspection of the differences between the estimated time effects from the “general relation” model (categorical time model described above) and those predicted by a linear regression of the effects on time.

The ALSFRS-R data is declared as non linear in the case that both criteria (statistical significance and major deviation as observed from the inspection described above) are met. In that case, the alternative analysis, proposed in hereinabove is used instead of the principal analysis described earlier.

Slope of the Changes from Baseline to Each Visit (In-Clinic and Telephonically) in ALSFRS-R Score (a Comparison Between 25 mg t.i.d. Talampanel and Placebo)

The adjusted mean change from baseline in ALSFRS-R across visits, derived from the treatment effect in the primary RMA of covariance model, is compared between the treatment groups. A decrease of 15% in the adjusted mean change from baseline in ALSFRS-R across visits of ALSFRS-R is observed in the group treated with talampanel as compared to the placebo group.

### Secondary Endpoint

Time from Baseline to the First Occurrence of Either Death, Tracheostomy or Permanent Assisted Ventilation (a Comparison Between 50 mg t.i.d. Talampanel and Placebo)

The time from baseline to the first of either death, tracheostomy, and permanent assisted ventilation, for all patients in the intent to treat (ITT) population is analyzed by the Cox proportional hazards model (SAS® PHREG procedure) to compare the risk of death/tracheostomy between treatment groups. Subjects with no reported death are assigned a right-censored value equal to the time that they were in the trial. The Kaplan-Meier estimates of the distribution of time to death are computed for each treatment group. The model includes country, Rilutek® use at baseline, site of ALS onset, time from ALS onset to baseline, baseline ALSFRS-R score, baseline Slow VC and baseline Body Mass Index (BMI) are included in the model. 50 mg t.i.d. of talampanel results in a reduction in the risk of either death, tracheostomy, or permanent assisted ventilation.

Time from Baseline to the First Occurrence of Either Death, Tracheostomy or Permanent Assisted Ventilation (a Comparison Between 25 mg t.i.d. Talampanel and Placebo)

The time from baseline to the first of either death, tracheostomy, and permanent assisted ventilation, for all patients in the intent to treat (ITT) population is analyzed by the Cox proportional hazards model (SAS® PHREG procedure) to compare the risk of death/tracheostomy between treatment groups. Subjects with no reported death are assigned a right-censored value equal to the time that they are in the trial. The Kaplan-Meier estimates of the distribution of time to death are computed for each treatment group. The model includes country, Rilutek® use at baseline, site of ALS onset, time from ALS onset to baseline, baseline ALSFRS-R score, baseline Slow VC and baseline Body Mass Index (BMI) are included in the model. 25 mg t.i.d. talampanel results in a reduction in the risk of either death, tracheostomy, or permanent assisted ventilation.

### Additional Endpoints

Change from Baseline to Last Observed Value (LOV) in ALSFRS-R

The adjusted means of the 50 mg t.i.d. group and the 25 mg t.i.d. group are compared to placebo by performing two relevant single degree of freedom comparisons. A decrease in the adjusted mean change from baseline to LOV in ALSFRS-R is observed in the group treated with talampanel as compared to the placebo group. A decrease in the change in ALSFRS-R from baseline to LOV is observed in the group treated with 50 mg t.i.d. talampanel in comparison to the placebo group.

### Time to Death

The analysis compares the treatment groups using the Cox's proportional hazard model (SAS® PHREG Procedure). The model includes the following terms: treatment group (placebo, 25 mg t.i.d. talampanel and 50 mg t.i.d. talampanel with placebo as reference category), center, Rilutek® use at baseline, site of ALS onset, time from ALS onset to baseline, baseline ALSFRS-R score, baseline slow VC and baseline BMI. The Kaplan-Meier estimates for the death are computed and presented in a graph for each treatment group. The group treated with 50 mg t.i.d. talampanel achieves a beneficial outcome as compared to the placebo group.

Slope of the Changes from Baseline to Each Visit in Pulmonary Function as Assessed by Slow VC

The statistical analysis is a comparison between the slopes of the treatment groups, derived from the time by treatment interaction term from the RMA model described above. The linearity of the SVC over time is tested with the same methodology as described for the primary endpoint.

A decrease in the slopes of Slow VC is observed in the group treated with 50 mg t.i.d. talampanel as compared to the placebo group.

Similarly, a decrease in the adjusted mean change from baseline in Slow VC across visits, derived from the treatment effect in the primary RMA of covariance model, is observed in the group treated with 50 mg t.i.d. talampanel as compares to the placebo group.

Change from Baseline to LOV in Slow VC

The change in Slow VC from baseline to LOV is compared between the treatment groups using ANCOVA (SAS® GLM procedure). Treatment group (placebo, 25 mg t.i.d. talampanel and 50 mg t.i.d. talampanel with placebo as reference category), center, Rilutek® use at baseline, age at baseline, site of ALS onset, time from ALS onset to baseline and baseline Slow VC value are included in the model. The group treated with 50 mg t.i.d. talampanel achieves a beneficial decrease in the change in Slow VC from baseline to LOV in comparison to the placebo group.

Slope of the Changes from Baseline to Each Visit in Manual Muscle Testing (MMT)

The analysis of the slopes of the MMT, based on the Repeated Measures analysis (RMA) of covariance model, comparing the treatment groups' slopes. The statistical analysis is a comparison between the treatment groups' slopes, derived from the time by treatment interaction term from the RMA model described above.

A decrease in the slopes of MMT is observed in the group treated with 50 mg t.i.d. talampanel as compared to the placebo group.

Similarly, a decrease in the adjusted mean change from baseline in MMT across visits, derived from the treatment effect in the primary RMA of covariance model, is observed in the group treated with 50 mg t.i.d. talampanel in comparison to the placebo group.

Change from Baseline to LOV in MMT

The change in MMT from baseline to LOV is compared between treatment groups using ANCOVA (SAS® GLM procedure). Treatment group (placebo, 25 mg t.i.d. talampanel and 50 mg t.i.d. talampanel), center, Rilutek® use at baseline, age at baseline, site of ALS onset, time from ALS onset to baseline and baseline MMT are included in the model.

A decrease in the change in MMT from baseline to LOV is observed in the group treated with talampanel as compared to the placebo group.

Change from Baseline to LOV in ALS-Specific Quality of Life (ALSSQoL) to Assess Drug Effect on Health Status

The change from baseline to LOV in ALSSQoL is compared between treatment groups using ANCOVA (SAS® GLM procedure). Treatment group (placebo, 25 mg t.i.d. talampanel and 50 mg t.i.d. talampanel), center, Rilutek® use at baseline, age at baseline, site of ALS onset, time from ALS onset to baseline and baseline ALSSQoL score are used as covariates. The group which is treated with 50 mg t.i.d. talampanel achieves a beneficial decrease in the change in ALSSQoL score from baseline to LOV in comparison to the placebo group.

Change from Baseline to LOV in Quality of Life EuroQol-5 Dimensions (EQ-5D) Health Outcomes Scale to Assess Drug Effect on Health Status

The change from baseline to LOV in a score is compared between treatment groups using ANCOVA (SAS® GLM procedure). Treatment group (placebo, 25 mg t.i.d. talampanel and 50 mg t.i.d. talampanel), center, Rilutek® use at baseline, age at baseline, site of ALS onset, time from ALS onset to baseline and baseline score are included in the model. The group which is treated with 50 mg t.i.d. talampanel achieves a beneficial decrease in the change in EQ-5D score from baseline to LOV in comparison to the placebo group.

Change from Baseline to LOV in Health Care Resource Utilization Questionnaire for ALS to Assess Drug Effect on Health Care Resource Use

Changes from baseline in each of the items in the questionnaire are analyzed using descriptive statistics presented by treatment group. The group which is treated with 50 mg t.i.d. talampanel achieves a beneficial decrease in the change in questionnaire score from baseline to LOV in comparison to the placebo group.

Change from Baseline to LOV in Zarit Burden Interview (ZBI) to Assess Drug Effect on Caregiver Burden

Change from baseline to LOV in ZBI score to assess drug effect on Caregiver Burden is compared between the treatment groups using ANCQVA (SAS® GLM procedure). Treatment group (placebo, 25 mg t.i.d. talampanel and 50 mg t.i.d. talampanel), center, Rilutek® use at baseline, age at baseline, site of ALS onset, time from ALS onset to baseline and baseline ZBI score are included in the model. The group which is treated with 50 mg t.i.d. talampanel achieves a beneficial decrease in the change in ZBI score from baseline to LOV in comparison to the placebo group.

### Dose Response Exploratory Analysis

Efforts are made to characterize the dose response curve, using exploratory analyses of the clinical data as well as the PK/PD relationship.

### Example 2

Another clinical trial is performed according to the procedure of Example 1 above and the results are obtained accordingly to preceding Example 1.

In this clinical trial the following results are:

### Primary Endpoint

Slope of the Changes from Baseline to Each Visit (In-Clinic and Telephonically) in ALSFRS-R Score (a Comparison Between 50 mg t.i.d. Talampanel and Placebo)

A statistically significant decrease of 20% in the slopes of ALSFRS-R is observed in the group treated with 50 mg t.i.d. talampanel as compared to the placebo group.

Slope of the Changes from Baseline to Each Visit (In-Clinic and Telephonically) in ALSFRS-R Score (a Comparison Between 25 mg t.i.d. Talampanel and Placebo)

A decrease of 20% in the slopes of ALSFRS-R is observed in the group treated with 25 mg t.i.d. talampanel as compared to the placebo group.

### Secondary Endpoint

Time from Baseline to the First Occurrence of Either Death, Tracheostomy or Permanent Assisted Ventilation (a Comparison Between 50 mg t.i.d. Talampanel and Placebo)

A decrease in the risk of either death, tracheostomy, or permanent assisted ventilation is observed in the group treated with 50 mg t.i.d. talampanel.

Time from Baseline to the First Occurrence of Either Death, Tracheostomy or Permanent Assisted Ventilation (a Comparison Between 25 mg t.i.d. Talampanel and Placebo)

25 mg t.i.d. talampanel results in a reduction in the risk of either death, tracheostomy, or permanent assisted ventilation.

### Additional Endpoints

Change from Baseline to Last Observed Value (LOV) in ALSFRS-R

A decrease of 20% in the change in ALSFRS-R from baseline to LOV is observed in the group treated with 50 mg t.i.d. talampanel in comparison to the placebo group.

Slope of the Changes from Baseline to Each Visit in Pulmonary Function as Assessed by Slow VC

A decrease in the slopes of Slow VC is observed in the group treated with 50 mg t.i.d. talampanel in comparison to the placebo group.

Similarly, a decrease in the adjusted mean change from baseline in Slow VC across visits, derived from the treatment effect in the primary RMA of covariance model, is observed in the group treated with 50 mg t.i.d. talampanel in comparison to the placebo group.

Slope of the Changes from Baseline to Each Visit in Manual Muscle Testing (MMT)

A decrease in the slopes of MMT are observed is the group treated with 50 mg t.i.d. talampanel as compared to the placebo group.

Similarly, a decrease in the adjusted mean change from baseline in MMT across visits, derived from the treatment effect is the primary RMA of covariance model, is observed in the group treated with 50 mg t.i.d. talampanel in comparison to the placebo group.

Change from Baseline to LOV in MMT

A decrease in the change in MMT from baseline to LOV is observed in the group treated with talampanel as compared to the placebo group.

Other parameters remain qualitatively similar to example 1.

### Example 3

Another clinical trial is performed according to the procedure of Example 1 above and the results are obtained accordingly to preceding Example 1.

In this clinical trial the following results are:

### Primary Endpoint

Slope of the Changes from Baseline to Each Visit (In-Clinic and Telephonically) in ALSFRS-R Score (a Comparison Between 50 mg t.i.d. Talampanel and Placebo)

A statistically significant decrease of 25% in the slopes of ALSFRS-R is observed in the group treated with 50 mg t.i.d. 20 talampanel as compared to the placebo group.

Slope of the Changes from Baseline to Each Visit (In-Clinic and Telephonically) in ALSFRS-R Score (a Comparison Between 25 mg t.i.d. Talampanel and Placebo)

A decrease of 25% in the slopes of ALSFRS-R is observed in the group treated with 25 mg t.i.d. talampanel as compared to the placebo group.

### Secondary Endpoint

Time from Baseline to the First Occurrence of Either Death, Tracheostomy or Permanent Assisted Ventilation (a Comparison Between 50 mg t.i.d. Talampanel and Placebo)

A decrease in the risk of either death, tracheostomy, or permanent assisted ventilation is observed in the group treated with 50 mg t.i.d. talampanel.

Time from Baseline to the First Occurrence of Either Death, Tracheostomy or Permanent Assisted Ventilation (a Comparison Between 25 mg t.i.d. Talampanel and Placebo)

25 mg t.i.d. talampanel results in a reduction in the risk of either death, tracheostomy, or permanent assisted ventilation.

### Additional Endpoints

Change from Baseline to Last Observed Value (LOV) in ALSFRS-R

A decrease of 25% in the change in ALSFRS-R from baseline to LOV is observed in the group treated with 50 mg t.i.d. talampanel in comparison to the placebo group.

Slope of the Changes from Baseline to Each Visit in Pulmonary Function as Assessed by Slow VC

A decrease in the slopes of Slow VC is observed in the group treated with 50 mg t.i.d. talampanel in comparison to the placebo group.

Similarly, a decrease in the adjusted mean change from baseline in Slow VC across visits, derived from the treatment effect in the primary RMA of covariance model, is observed in the group treated with 50 mg t.i.d. talampanel in comparison to the placebo group.

Slope of the Changes from Baseline to Each Visit in Manual Muscle Testing (MMT)

A decrease in the slopes of MMT is observed in the group treated with 50 mg t.i.d. talampanel as compared to the placebo group.

Similarly, a decrease in the adjusted mean change from baseline in MMT across visits, derived from the treatment effect in the primary RMA of covariance model, is observed in the group treated with 50 mg t.i.d talampanel in comparison to the placebo group.

Change from Baseline to LOV in MMT

A decrease in the change in MMT from baseline to LOV is observed in the group treated with talampanel as compared to the placebo group.

Other parameters remain qualitatively similar to example 1.

### Example 4

Another clinical trial is performed according to the procedure of Example 1 above and the results are obtained accordingly to preceding Example 1.

In this clinical trial the following results are:

### Primary Endpoint

Slope of the Changes from Baseline to Each Visit (In-Clinic and Telephonically) in ALSFRS-R Score (a Comparison Between 50 mg t.i.d. Talampanel and Placebo)

A statistically significant decrease of 30% in the slopes of ALSFRS-R is observed in the group treated with 50 mg t.i.d. talampanel as compared to the placebo group.

Slope of the Changes from Baseline to Each Visit (In-Clinic and Telephonically) in ALSFRS-R Score (a Comparison Between 25 mg t.i.d. Talampanel and Placebo)

A decrease of 30% in the slopes of ALSFRS-R is observed in the group treated with 25 mg t.i.d. talampanel as compared to the placebo group.

### Secondary Endpoint

Time from Baseline to the First Occurrence of Either Death, Tracheostomy or Permanent Assisted Ventilation (a Comparison Between 50 mg t.i.d. Talampanel and Placebo)

A decrease in the risk either death, tracheostomy or permanent assisted ventilation is observed in the group treated with 50 mg t.i.d. talampanel.

Time from Baseline to the First Occurrence of Either Death, Tracheostomy or Permanent Assisted Ventilation (a Comparison Between 25 mg t.i.d. Talampanel and Placebo)

25 mg t.i.d. talampanel results in a reduction in the risk of either death, tracheostomy, or permanent assisted ventilation.

### Additional Endpoints

Change from Baseline to Last Observed Value (LOV) in ALSFRS-R

A decrease of 30% in the change in ALSFRS-R from baseline to LOV is observed in the group treated with 50 mg t.i.d. talampanel in comparison to the placebo group.

Slope of the Changes from Baseline to Each Visit in Pulmonary Function as Assessed by Slow VC

A decrease in the slopes of Slow VC is observed in the group treated with 50 mg t.i.d. talampanel in comparison to the placebo group.

Similarly, a decrease in the adjusted mean change from baseline in Slow VC across visits, derived from the treatment effect in the primary RMA of covariance model, is observed in the group treated with 50 mg t.i.d. talampanel in comparison to the placebo group.

Slope of the Changes from Baseline to Each Visit in Manual Muscle Testing (MMT)

A decrease in the slopes of MMT is observed in the group treated with 50 mg t.i.d. talampanel as compared to the placebo group.

Similarly, a decrease in the adjusted mean change from baseline in MMT across visits, derived from the treatment effect in the primary RMA of covariance model, is observed in the group treated with 50 mg t.i.d. talampanel in comparison to the placebo group.

Change from Baseline to LOV in MMT

A decrease in the change in MMT from baseline to LOV is observed in the group treated with talampanel as compared to the placebo group.

Other parameters remain qualitatively similar to example 1.

### Conclusions

The instant invention provides, for the first time, evidence of the efficacy, tolerability and safety of 150 mg daily doses of talampanel in subjects with ALS. Specifically, the results described herein show that talampanel reduces the rate of functional decline and the progression of disease symptoms in ALS patients, decreases the incidence of death, tracheostomy, or permanent assisted ventilation in treated patients as compared to patients receiving placebo, and is well tolerated and safe to use. The results provided herein thus substantiate, for the first time, the use of 150 mg total daily dosage of talampanel for treating human patients suffering from Amyotrophic Lateral Sclerosis.

### Statistical Analysis

The overall significance level for this study is 5% using two-tailed tests. The hierarchy approach is adopted to maintain the overall study type I error, where each endpoint contrast is analyzed only in case that the preceding endpoint contrast (according to the following order) p-value is less than 0.05. The hierarchy order is as follows:
Contrast 1: The primary endpoint: The 50 mg t.i.d. talampanel group and the placebo group contrast.
Contrast 2: The secondary endpoint: The 50 mg t.i.d. talampanel group and the placebo group contrast.
Contrast 3: The primary endpoint: The 25 mg t.i.d. talampanel group and the placebo contrast.
Contrast 4: The secondary endpoint: The 25 mg t.i.d. talampanel group and the placebo contrast.

The principal statistical analysis of the primary endpoint employs Repeated Measures Analysis (RMA) of covariance model (SAS® MIXED procedure). The analysis, aims to compare the ALSFRS-R slopes of change from baseline between the 50 mg t.i.d. talampanel group and the placebo group, use as the dependent variable the subject's computed change from baseline in ALSFRS-R at each of the monthly post-randomizations visits (in-clinic and telephonically). The model includes the following fixed effects: time from baseline, treatment group (placebo, 25 mg t.i.d. talampanel and 50 mg t.i.d. talampanel), time by treatment interaction, center, riluzole use at baseline (no/yes), age at baseline, site of ALS onset, time from ALS onset to baseline and baseline ALSFRS-R score. The individual subject intercept and time effects are also included in the model as random effects (random intercept and slope model). The principal statistical analysis compares the slopes of 50 mg t.i.d. talampanel and placebo groups, derived from the relevant contrast defined for the time by treatment interaction term in the model described above. The contrast that compares the slope between the 25 mg talampanel group and placebo group is evaluated according to the hierarchy approach defined above.

The principal analysis is based on the assumption of linear decline in ALSFRS-R within treatment group, a pattern that has been shown previously in other ALS studies. Nevertheless, both statistical testing and visual inspection of ALSFRS-R means are performed. To test if the relation between ALSFRS-R and time-in-trial is linear within treatment groups, a repeated measures ANCOVA model (SAS® MIXED procedure) is fitted, similar to the one proposed for the principal analysis, first with time as a continuous variable (representing a linear relation), and second, with time as a by-month categorical variable (representing a general relation). The log likelihood ratio test for nested models is used to test the contribution of the “general relation” beyond the “linear relation”. Statistically significant deviation from linearity is declared if the p-value of the chi-square statistic with the appropriate degrees of freedom is be less than 0.05.

Due to the high sensitivity of this test, small deviations may yield statistical significance, although clinically irrelevant. Therefore, to determine if the deviation is clinically meaningful, a visual inspection of the mean changes in ALSFRS-R by time, separately for each group, is performed, as well as inspection of the differences between the estimated time effects from the “general relation” model (categorical time model described above) and those predicted by a linear regression of the effects on time.

The ALSFRS-R data is declared as non linear in the case that both criteria (statistical significance and major deviation as observed from the inspection described above) are met. In that case, an alternative analysis is used instead of the principal analysis described above.

If the hypothesis of linearity within treatment groups is rejected, the change in ALSFRS-R from baseline to last observed value (LOV) is compared between the 50 mg t.i.d. talampanel group and the placebo group using an analysis of covariance (ANCOVA, SAS® GLM procedure) with the relevant contrast. The model includes the following terms: treatment group (placebo, 25 mg t.i.d. talampanel and 50 mg t.i.d. talampanel), center, riluzole use at baseline, age at baseline, site of ALS onset, time from ALS onset to baseline and baseline ALSFRS-R score. The adjusted means of the 50 mg t.i.d. and placebo are compared by performing a relevant contrast. To check the robustness of this analysis to the possible biases due to dropouts, Multiple Imputation Analysis is performed (SAS® MI procedure). The contrast that compares the 25 mg talampanel group and placebo group is evaluated according to the hierarchy approach defined above.

Treatment by center interaction is tested in the primary analysis (or alternative, if applied) at a significance level of 10%. The test aims to assess different slope differences between centers; therefore is based on the three order treatment by time by center interaction. The test is based on log likelihood ratio test between a model similar to the proposed for the principal analysis and the same model including the three-order interaction. The second order center by treatment interaction is included in both models. If the three order treatment by time by center interaction is found significant, the component due to the interaction between center and the 50 mg t.i.d. talampanel/placebo contrast is identified. If the latter is not significant, no attempt is made to further investigate the source of interaction. If the number of subjects in some of the centers is small, the heterogeneity of treatment effects is explored by pooling of centers (using country instead of center).

### Discussion

At present, the only known effective treatment for ALS involves modulation of glutamate activity. In multiple preclinical studies, it has become apparent that the most likely target for glutamate excitotoxicity in ALS is the AMPA receptor. Multiple selective AMPA antagonists have shown to delay disease progression in the transgenic animal ALS model. Talampanel is a selective AMPA receptor antagonist.

The AMPA (α-amino-3-hydroxy-5-methyl-4-isoxazolepropionic acid) subtype of glutamate receptor is responsible for much of the fast excitatory transmission in the mammalian CNS. The excitatory transmission process is terminated by active re-uptake of glutamate from the synaptic cleft by several transporter proteins located on both neuronal and glial cells (Rothstein J D, Dykes-Hoberg M, Pardo C A, Bristol L A, et al. Knockout of glutamate transporters reveals a major role for astroglial transport in excitotoxicity and clearance of glutamate. Neuron 1996; 16: 675-686.), (Rothstein J D, Jin L, Dykes-Hoberg M, Kuncl R W. Chronic inhibition of glutamate uptake produces a model of slow neurotoxicity. Proc Natl Acad Sci USA 1993; 90: 6591-6595). If, however, there is some disturbance to the normal excitatory transmission process that leads to over-stimulation of glutamate receptors, there may be an excessive influx of calcium. Calcium entry via glutamate receptor ion channels is one of the key processes for triggering toxic events within the cell and eventual neuronal damage. In areas of the cortex affected in ALS there is evidence of reduced levels of expression of the glial glutamate transporter and reduced glutamate uptake activity (Rothstein J D, Martin L J, Kunel R W. Decreased glutamate transport by the brain and spinal cord in amyotrophic lateral sclerosis. N Engl J Med 1992; 326: 1464-1468).

AMPA receptors are composed of four protein subunits, GluR1-GluR4. The GluR2 subunit is of particular functional significance because its presence renders the AMPA receptor impermeable to calcium. Most AMPA receptors in the human CNS include the edited form of the GluR2 subunit and are calcium impermeable. However, upper and lower motor neurons have low expression of mRNA for the GluR2 AMPA receptor subunit. This low expression implies that most of the surface AMPA receptors on human motor neurons are likely to be atypical and calcium permeable. This feature may explain why motor neurons appear to be particularly vulnerable to the excitotoxic effects of glutamate. Indeed, studies have shown that there is a defect in the editing of the messenger RNA encoding the GluR2 subunit of glutamate AMPA receptors in the spinal motor neurons of individuals affected by ALS (Nature. 2004 Feb. 26; 427(6977) :801).

Talampanel is an orally active noncompetitive antagonist (or negative allosteric modulator) of the AMPA α-amino-3-hydroxy5-methyl-4-isoxazolepropionic acid) subtype of glutamate excitatory amino acid receptors. The prototype compound for talampanel is GYKI-52466, a 2,3-benzodiazepine with no classical 1,4-benzodiazepine (that is, “diazepam-like”) activity at the GABA receptor complex (Tarnawa I, Farkas S, Berzsenyi P, Pataki A, Andrasi F. Electrophysiological studies with a 2,3-benzodiazepine muscle relaxant: GYKI 52466. Eur J Pharmacol 1989; 167: 193-199). Talampanel is a more potent analog of GYKI-52466.

Numerous research papers have been published on GYKI-52466, the prototype compound of this class. Talampanel is a more potent analog of GYKI-52466. The literature reports on the pharmacology of GYKI-52466 and the preclinical studies of talampanel document the potent and selective AMPA antagonist activity of these compounds in both in vitro and in vivo perpetrations. Talampanel has also been studied in preclinical tests predictive of efficacy in models of neurodegeneration that predict efficacy as a neuroprotectant. Glutamate is the predominant neurotransmitter used in the mammalian brain for excitatory neurotransmission. Prolonged excitatory neurotransmission, however, can even lead to overt neuronal degeneration through a mechanism known as glutamate excitotoxicity, a process implicated in a variety of acute and chronic neurodegenerative disorders. Because normal, excessive and excitotoxic glutamate transmission lie on a single continuum, a glutamate receptor antagonist which limits excursions through this continuum may have multiple therapeutic opportunities. Data from both in vitro and in vivo experiments revealed that talampanel blocks or prevents excessive excitotoxic glutamate transmission.

Many causes of ALS have been proposed including toxicity from excess excitation of the motor neuron by transmitters such as glutamate free radical-mediated oxidative cytotoxicity, protein aggregation, neuroinflammation, mitochondrial dysfunction, auto-immune processes and cytoskeletal abnormalities. Superoxide dismutase (SOD), an enzyme important in reducing cellular levels of the superoxide free radical, has been linked to familial ALS. However, mutations in the gene encoding SODl account for only about 20% of the familial cases of ALS, or 2% of all ALS (Rosen D Siddique T, Patterson D, Figlewicz D, et al. Mutations in Cu/Zn superoxide dismutase gene are associated with familial amyotrophic lateral sclerosis. Nature 1993; 362: 59-62).

The excitatory neurotransmitter, glutamate, is widely distributed in the mammalian CNS. An excess of glutamate in the extracellular environment has been shown to result in neuronal death. The process is known as ‘excitotoxicity’, and is based on altered extracellular concentrations of glutamate. Normally, low levels of glutamate are maintained in the extracellular environment by active uptake mechanisms present on neurons and glia (Rothstein J D, Dykes-Hoberg M, Pardo C A, Bristol L A, et al. Knockout of glutamate transporters reveals a major role for astroglial transport in excitotoxicity and clearance of glutamate. Neuron 1996; 16: 675-686), (Skradski S, White H. Topiramate blocks kainate-evoked cobalt influx into cultured neurons. Epilepsia 2000; 41(Suppl 1): S45-S47).

Several lines of evidence suggest a role for glutamate in the pathogenesis of ALS (Gurney M E, Cutting F B, Zhai P, Andrus P K, et al. Pathogenic mechanisms in familial amyotrophic lateral sclerosis due to mutation of Cu, Zn superoxide dismutase. Pathol Biol (Paris) 1996; 44: 51-56), (Plaitakis A, Constantakakis E. Altered metabolism of excitatory amino acids, N-acetyl-aspartate and N-acetyl-aspartyl-glutamate in amyotrophic lateral sclerosis. Brain Res Bull 1993; 30: 381386), (Rothstein J D, Martin L J, Kuncl R W. Decreased glutamate transport by the brain and spinal cord in amyotrophic lateral sclerosis. N Engl J Med 1992; 326: 1464-1468). Increased glutamate levels have been demonstrated in the CSF of ALS patients. Reduced glutamate transport was found in synaptosomes derived from spinal cord and motor cortex of ALS patients but not other brain areas (Feldmeyer D, Kask K Brusa R, Kornau H C et al. Neurological dysfunctions in mice expressing different levels of the Q/R site-unedited AMPAR subunit GluR-B. Nat Neurosci 1999; 2: 57-64). In an organotypic spinal cord slice model, reduction in glutamate uptake results in motor neuron death (Rothstein J D, Jin L, Dykes-Hoberg M, Kuncl R W. Chronic inhibition of glutamate uptake produces a model of slow neurotoxicity. Proc Natl Acad Sci USA 1993; 90: 6591-6595) and a variety of glutamate antagonists inhibit this process (Bilak M M, Corse A M, Kuncl R W., Additivity and potentiation of IGF-I and GDNF in the complete rescue of postnatal motor neurons., Amyotroph Lateral Scler Other Motor Neuron Disord 2001; 2: 83-91), (Drachman D B, Rothstein J D. Inhibition of cyclooxygenase-2 protects motor neurons in an organotypic model of amyotrophic lateral sclerosis. Ann Neurol 2000; 48, 792-795), (Ho T W, Bristol L A, Coccia C, Li Y, et al. TGFbeta trophic factors differentially modulate motor axon outgrowth and protection from excitotoxicity. Exp Neurol 2000; 161: 664-675), (Maragakis N J, Jackson M, Ganel R, Rothstein J D. Topiramate protects against motor neuron degeneration in organotypic spinal cord cultures but not in G93A SOD1 transgenic mice. Neurosci Lett 2003; 338: 107-110).

Motor neurons appear to be particularly vulnerable to excitotoxic effects of glutamate and this is mediated by the glutamate AMPA receptor (Carriedo S G, Sensi S L, Yin H Z, Weiss J H. AMPA exposures induce mitochondrial Ca (2+) overload and ROS generation in spinal motor neurons in vitro. J Neurosci 2000; 20: 240-250), (Hugon J, Vallat J M, Dumas M. [Role of glutamate and excitotoxicity in neurologic diseases]. Rev Neurol (Paris) 1996; 152: 239-248), (Ikonomidou C, Qin Qin Y, Labruyere J, Olney J W. Motor neuron degeneration induced by excitotoxin agonists has features in common with those seen in the SOD-1 transgenic mouse model of amyotrophic lateral sclerosis. J Neuropathol Exp Neurol 1996; 55: 211-224), (Saroff D, Delfs, J, Kuznetsov D, Geula C. Selective vulnerability of spinal cord motor neurons to non-NMDA toxicity. Neuroreport 2000; 11: 1117-1121), (Skradski S, White H. Topiramate blocks kainate-evoked cobalt influx into cultured neurons. Epilepsia 2000; 41(Suppl 1): S45-S47

The selective susceptibility of motor neurons may be due to the fact that the AMPA receptor subtype present on motor neurons does not gate calcium entry. Calcium influx through glutamate AMPA receptors appears to be a major trigger for motor neuron death (Bar-Peled 0, O'Brien R J, Morrison J H, Rothstein J D. Cultured motor neurons possess calcium-permeable AMPA/kainate receptors. Neuroreport 1999 10: 855-859), (Williams T L, Day N C, Ince P G, Kamboj R K, et al. Calcium-permeable alpha-amino3-hydroxy-5-methyl-4˜isoxazole propionic acid receptors: a molecular determinant of selective vulnerability in amyotrophic lateral sclerosis. Ann Neurol 1997; 42: 200-207), (The ALS CNTF Treatment Study (ACTS) Phase I-II Study Group. The Amyotrophic Lateral Sclerosis Functional Rating Scale: assessment of activities of daily living in patients with amyotrophic lateral sclerosis. Arch Neurol 1996; 53: 141-147). In support of this, transgenic mice overexpressing a glutamate receptor (GluR-B subunit) that is highly permeable to calcium display a motor neuron syndrome late in life (Feldmeyer D, Kask K, Brusa R, Kornau H C, et al. Neurological dysfunctions in mice expressing different levels of the Q/R site-unedited AMPAR subunit GluR-B. Nat Neurosci 1999; 2: 57-64).

In vivo mouse and, human studies support the idea that glutamate excitotoxicity in general, and AMPA mediated glutamate effects in particular, may be an important target for potential therapeutic agents in ALS. Rilutek®, the single FDA approved treatment for ALS, acts in a variety of ways to reduce glutamate release and to antagonize its activity. Topiramate, an anticonvulsant with anti-excitotoxic properties, diminishes glutamate release from neurons, and antagonizes kainate excitation via glutamate AMPA receptors (Skradski S, White H. Topiramate blocks kainate-evoked cobalt influx into cultured neurons. Epilepsia 2000; 41(Suppl 1): S45-S47). While topiramate protected motor neurons in an organotypic spinal cord slice model, in clinical trial in ALS patients no benefit, was demonstrated (Cudkowicz M E, Shefner J M, Schoenfeld D A, Brown Jr R H, Jr., et al. A randomized, placebo-controlled trial of topiramate in amyotrophic lateral sclerosis. Neurology 2003; 61: 456-464). The lack of benefit was suggested to be dose related; as significant adverse events were noted in patients, including dramatic weight loss. In the mouse model of ALS, AMPA antagonists have shown clear beneficial effects. The selective AMPA antagonist NBQX blocked kainate induced calcium entry into motor neurons in vitro, and prolonged survival in ALS mice by 14 days (Van Damrne P, Leyssen M, Callewaert G, Robberecht W, et al. The AMPA receptor antagonist NBQX prolongs survival in a transgenic mouse model of amyotrophic lateral sclerosis. Neurosci Lett 2003; 343: 81-84). Similarly, another selective AMPA antagonist RPR: 119990, prolonged life and maintained muscle strength in, a dose dependent fashion, with life expectancy prolonged by 20 days at the highest dose used (Canton T, Bohme G A, Boireau A, Bordier F, et al. RPR 119990, a novel alpha-amino-3-hydroxy-5-methyl-4-isoxazolepropionic acid antagonist: synthesis, pharmacological properties, and activity in an animal model of amyotrophic lateral sclerosis. J Pharmacol Exp Ther 2001; 299: 314-322).

Based on the above, it is clear that drugs that interfere with the glutamate excitatory transmission process by reducing glutamate release or by antagonizing its activity are potential candidates in the treatment of ALS. Talampanel is an AMPA receptor antagonist inhibiting glutamate influx by AMPA receptor on the postsynaptic terminal, therefore affecting excitotoxicity but by a different mode of action to that of Rilutek®.

1. A method of treating a human patient afflicted with amyotrophic lateral sclerosis (ALS) comprising periodically administering to the human patient for a therapeutically effective duration a pharmaceutical composition comprising an amount of talampanel therapeutically effective to provide a benefit to the human patient, thereby treating the human patient.

2. The method of claim 1, wherein the benefit is a reduction in the rate of decrease in the ALSFRS-R score, the Manual Muscle Testing (MMT) score, the Slow Vital Capacity (VC) percent predicted value, the ALS-Specific Quality of Life (ALSSQoL) score, EuroQol-5 Dimensions (EQ-5D) Health Outcomes Scale score.

3. The method of claim 1, wherein the benefit is a reduction in the rate of increase in the Zarit Burden Interview (ZBI) score.

4. The method of claim 1, wherein the benefit is a reduction in the rate of decrease in the Manual Muscle Testing (MMT) score, the Slow Vital Capacity (VC) percent predicted value, ALS-Specific Quality of Life (ALSSQoL) score, EuroQol-5 Dimensions (EQ-5D) Health Outcomes Scale score.

5. A method of treating a symptom of amyotrophic lateral sclerosis (ALS) in a human patient afflicted with ALS comprising periodically administering to the human patient for a therapeutically effective duration a pharmaceutical composition comprising an amount of talampanel therapeutically effective to alleviate the symptom of ALS in the human patient.

6. The method of claim 5, wherein the symptom of ALS is measured by the ALSFRS-R scale, the Manual Muscle Testing (MMT) grading system, the Slow Vital Capacity (VC) assay, the ALS-Specific Quality of Life (ALSSQoL) scale, EuroQol-5 Dimensions (EQ-5D) Health Outcomes Scale, Health Care Resource Utilization Questionnaire for ALS scale or Caregiver burden questionnaire, Zarit Burden Interview (ZBI) scale.

7. The method of claim 5, wherein the symptom of ALS is measured by the Manual Muscle Testing (MMT) grading system, the Slow Vital Capacity (VC) assay, the ALS-Specific Quality of Life (ALSSQoL) scale, EuroQol-5 Dimensions (EQ-SD) Health Outcomes Scale, Health Care Resource Utilization Questionnaire for ALS scale or Caregiver burden questionnaire, Zarit Burden Interview (ZBI) scale.

8. The method of claim 2, wherein the rate of decrease in, or the symptom as measured by, the ALSFRS-R scale, is reduced by at least 15% as compared to a human patient not administered the pharmaceutical composition.

9. The method of claim 1, wherein the benefit or the symptom is measured by an ALS validated outcome measure assessment protocol.

10. The method of claim 1, wherein the duration of administration is greater than thirty-six weeks.

11. The method of claim 10, wherein the duration of administration is 52 weeks.

12. The method of claim 11, wherein the periodic administration is three times per day.

13. The method of claim 12, wherein the therapeutically effective amount of talampanel per administration is 25 mg.

14. The method of claim 12, wherein the therapeutically effective amount of talampanel per administration is 50 mg.

15. The method of claim 12 wherein the therapeutically effective amount of talampanel is 75 mg/day.

16. The method of claim 12 wherein the therapeutically effective amount of talampanel is 150 mg/day.

17. The method of claims 12 further comprising escalating a total daily dose administered to the human patient.

18. The method of claim 17, wherein the total daily dose is escalated from 37.5 mg to 75 mg.

19. The method of claim 18, wherein the escalating step comprises administering 37.5 mg total daily dose from baseline visit to week 1, 50 mg total daily dose from week 1 to week 2, 62.5 mg total daily dose from week 2 to week 3, and 75 mg total daily dose from week 3 and thereafter.

20-23. (canceled)

24. The method of claim 17, wherein the total daily dose is escalated from 75 mg to 150 mg.

25. The method of claim 24, wherein the escalating step comprises administering 75 mg total daily dose from baseline visit to week 1, 100 mg total daily dose from week 1 to week 2, 125 mg total daily dose from week 2 to week 3, and 150 mg total daily dose from week 3 and thereafter.

26-36. (canceled)

37. A method for reducing the rate of functional decline in a human patient afflicted with amyotrophic lateral sclerosis (ALS) comprising periodically administering to the human patient for a therapeutically effective duration a pharmaceutical composition comprising an amount of talampanel therapeutically effective to reduce the rate of functional decline in the human patient.

38-43. (canceled)
